(19) United States
(12) Patent Application Publication
(10) Pub. No.: US 2020/0223779 A1
(43) Pub. Date: Jul. 16, 2020

(54) CATALYTIC CYCLE FOR PRODUCTION OF 1,1-DISUBSTITUTED ALKENES

(71) Applicant: Sirrus, Inc., Loveland, OH (US)

(72) Inventors: Jeffrey M. Sullivan, Cincinnati, OH (US); Ami Doshi, Loveland, OH (US); Kshitij Kishen Parab, Loveland, OH (US)

(21) Appl. No.: 16/641,415

(22) PCT Filed: Sep. 12, 2018
(86) PCT No.: PCT/US2018/050540

§ 371 Date: Feb. 24, 2020

Related U.S. Application Data
(60) Provisional application No. 62/560,280, filed on Sep. 19, 2017.

(51) Int. Cl. C07C 67/30; C07C 45/61; B01J 31/04

(52) U.S. Cl. CPC C07C 67/30; C07C 45/61; B01J 2231/4288; B01J 2531/002; B01J 31/04

(57) ABSTRACT

The present teachings disclose contacting an amine salt catalyst with a dicarbonyl compound having an alkylene group between the carbonyl group; adding formaldehyde, paraformaldehyde, or formalin in an amount of about 2:1 to about 3:1 moles of formaldehyde to moles of the dicarbonyl compound to form a mixture; and refluxing the mixture. The process forms a carbonyl-substituted alkene. The process may be performed in the absence of a solvent. The process may form methylene malonates, methylene dimalonates, methylene keto malonamides, methylene diketones, methylene keto esters, and the like.

### CROSS-REFERENCE TO RELATED APPLICATIONS

The present application claims priority under 35 U.S.C. 119(e) to U.S. Provisional Application Ser. No. 62/560,280, filed Sep. 19, 2017, which is hereby incorporated by reference in its entirety for all purposes.

### FIELD

Disclosed are processes for the preparation of a carbonyl-substituted alkene, such as 1,1-disubstituted alkenes. The processes disclosed eliminate an intermediate diol and a diol cracking step.

### BACKGROUND

1,1-disubstituted alkenes are of interest because they are capable of polymerizing at ambient temperatures with contact with basic materials. In addition, their functional groups provide great flexibility in forming a variety of compounds and polymerizable compositions. 1,1-disubstituted alkenes include methylene malonates, methylene dimalonamides, methylene keto malonamides, methylene diketones, methylene keto esters, and the like. Such compounds have been known since 1886 where the formation of diethyl methylene malonate was first demonstrated by W. H. Perkin, Jr. (Perkin, Ber. 19, 1053 (1886)). The early methods for producing methylene malonates suffer significant deficiencies that preclude their use in obtaining commercially viable monomers, including unwanted polymerization of the monomers during synthesis (e.g., formation of polymers or oligomers or alternative complexes), formation of undesirable side products (e.g., ketals or other latent acid-forming species which impede rapid polymerization), degradation of the product, insufficient and/or low yields, and ineffective and/or poorly functioning monomer product (e.g., poor adhesive characteristics, stability, or other functional characteristics), among other problems. The overall poorer yield, quality, and chemical performance of the monomer products formed by prior methods have impinged on their practical use in the production of the above products.

In recent years a number of commonly owned patent applications have been filed which have solved a number of the problems associated with the synthesis of methylene malonates and analogs thereof, for example, Malofsky et al., U.S. Pat. No. 8,609,885; Malofsky, U.S. Pat. No. 8,884,051; Malofsky et al., U.S. Pat. No. 9,108,914; and Sullivan et al., U.S. Pat. No. 9,518,001. The synthesis procedures provided therein result in improved yields of heretofore elusive, high quality methylene malonates and other polymerizable compositions.

Malofsky et al., U.S. Pat. No. 9,108,914 discloses preparing methylene malonates in a two-step process where the first step comprises reacting a source of formaldehyde with a dialkyl malonate ester in the presence of a reaction catalyst to form a diol reaction product comprising the dialkyl bis(hydroxymethyl) malonate composition; and reacting a dialkyl bis(hydroxyl-methyl) malonate composition in the presence of a suitable catalyst to form a methylene malonate monomer and isolating the methylene malonate monomer. The disclosed catalysts are bases, such as calcium hydroxide exemplified, which need to be removed before the second step to avoid unwanted polymerization of the 1,1-dicarbonyl substituted-1-ethylenes and methylene malonates.

Sullivan et al., U.S. Pat. No. 9,518,001 also discloses preparing methylene malonates, through the formation of an intermediate reaction product and hydroxyl methyl groups undergoing a “cracking reaction,” referring to the thermolysis of a 1,1-dicarbonyl substituted-1,1-bis (hydroxymethyl)-methanes to a monomer species with the release of formaldehyde and water.

Industry is constantly seeking new, or simpler ways, of producing carbonyl-substituted alkenes. It is desirable to provide a general synthetic route to a variety of 1,1-disubstituted alkenes, such as methylene malonates, methylene dimalonamides, methylene keto malonamides, methylene diketones, methylene keto esters, and the like. There is also a desire to provide a direct route to these 1,1-disubstited alkenes, thereby eliminating an intermediate reaction product (e.g., an intermediate diol) and the need for a cracking reaction or cracking step.

Efforts to provide a direct methylenation of carbonyls are described in an article entitled “Efficient, direct α-methylenation of carbonyls mediated by diisopropylammonium trifluoroacetate” (46 Chem. Commun. 2010, 1715-17). However, the reactions disclosed therein require a large excess of paraformaldehyde, and the reaction time is long (i.e., over 8 hours). The article also notes that reactions were significantly slower when a catalytic amount of the catalyst was used. A solvent is required, with the catalyst used in stoichiometric amounts. It is desirable to reduce the amount of formaldehyde required for the reaction, reduce the reaction time, reduce the amount of catalyst needed, perform the reaction in the absence of a solvent, with the catalyst used in a catalytic amount, or a combination thereof.

Thus, what is needed is an improved or simplified process that can prepare carbonyl-substituted alkenes, such as 1,1-disubstituted alkenes. What is needed is a general synthetic route to all 1,1-disubstituted alkenes, such as methylene malonates, methylene dimalonamides, methylene keto malonamides, methylene diketones, methylene keto testers, and the like. What is also needed is a direct route to 1,1-disubstituted alkenes, thereby eliminating an intermediate diol and eliminating the diol cracking step. What is further needed is a process that minimizes steps for preparing these 1,1-disubstituted alkenes, reduces waste, reduces the number of components or amounts of ingredients, allows for a solvent-free process, has a faster reaction time, or a combination thereof.

### SUMMARY

Disclosed is a process comprising: contacting an amine salt catalyst with a dicarbonyl compound having an alkylene group between the carbonyl groups; adding formaldehyde, paraformaldehyde, or formalin in an amount of about 2:1 to about 3:1 moles of formaldehyde to moles of the dicarbonyl compound to form a mixture; and refluxing the mixture; wherein the process forms a carbonyl-substituted alkene. The process may be a Mannich type reaction. The process may form methylene malonates, methylene dimalonates, methylene keto malonamides, methylene diketones, methylene keto esters, and the like.

The dicarbonyl compound may be a diester, diketone, diamide, ketoester, ketoamide or ester amide. The dicarbonyl compound may be a hydrocarbyl malonate, where the hydrocarbyl group is an alkyl, cycloalkyl, or polyether group. The dicarbonyl compound may be a diketone with one or more aryl substituted alkyl groups. The amine salt catalyst may be prepared by reacting an acid with a base. The acid may have a Bronsted acidity of about 2 to about −5. The acid may have a pKa of about 2 to about −5. Exemplary acids may be selected from trifluoracetic acid, sulfuric acid, or methanesulfonic acid. The base may be a sterically hindered ammonium cation. The base may be a secondary ammonium cation. The amine salt catalyst may be diisopropylammonium trifluoroacetate. The mixture of the process may be heated to a temperature of about 80° C. or greater. The mixture of the process may be heated to a temperature of about 120° C. or less. The process may have a reaction time of about 5 hours or less, or about 3 hours or less. The process may achieve a yield of about 90% or greater.

The process may be performed in the absence of a solvent. The amine salt catalyst may be supplied in a catalytic amount. For example, the amine salt catalyst may be supplied in an amount of about 25 mol % or less, about 10 mol % or less, or about 7 mol % or less based on the dicarbonyl compound.

The process may be performed using a solvent. The process may include dissolving the dicarbonyl compound in a solvent, such as a polar aprotic solvent. The amine salt catalyst may be supplied in a stoichiometric amount.

### DETAILED DESCRIPTION

The explanations and illustrations presented herein are intended to acquaint others skilled in the art with the invention, its principles, and its practical application. The specific embodiments of the present disclosure as set forth are not intended to be exhaustive or limit the scope of the disclosure. The scope of the disclosure should be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

Disclosed are processes for a direct route to 1,1-disubstituted alkenes. The process may include contacting an amine salt catalyst with a dicarbonyl compound having an alkylene group between the carbonyl groups; adding a formaldehyde source in an amount of about 2:1 to about 3:1 moles of formaldehyde to moles of the dicarbonyl compound to form a mixture; and refluxing the mixture; where the process forms a carbonyl-substituted alkene. The process may be a Mannich type reaction, which may result in producing methylene malonates, methylene dimalonamides, methylene ketomalonamides, methylene diketones, methylene ketoesters, and the like, without requiring an intermediate diol product or a diol cracking step. A Mannich reaction is an aminoalkylation reaction involving the condensation of an enolizable carbonyl compound with a nonenolizable aldehyde and a primary or a secondary amine to produce a β-aminocarbonyl compound.

One or more as used herein means that at least one, or more than one, of the recited components may be used as disclosed. Nominal as used with respect to functionality means the theoretical functionality, generally this can be calculated from the stoichiometry of the ingredients used. Generally, the actual functionality is different due to imperfections in raw materials, incomplete conversion of the reactants and formation of by-products. Residual content of a component refers to the amount of the component present in free form or reacted with another material, such as an oligomer or a polymer. Typically, the residual content of a component can be calculated from the ingredients utilized to prepare the component or composition. Alternatively, it can be determined utilizing known analytical techniques. Heteroatom means nitrogen, oxygen, sulfur and phosphorus. In some embodiments, heteroatoms are nitrogen and oxygen. Hydrocarbyl as used herein refers to a group containing one or more carbon atom backbones and hydrogen atoms, which may optionally contain one or more heteroatoms. Where the hydrocarbyl group contains heteroatoms, the heteroatoms may form one or more functional groups well known to one skilled in the art. Hydrocarbyl groups may contain cycloaliphatic, aliphatic, aromatic, or any combination of such segments. The aliphatic segments can be straight or branched. The aliphatic and cycloaliphatic segments may include one or more double and/or triple bonds. Included in hydrocarbyl groups are alkyl, alkenyl, alkynyl, aryl, cycloalkyl, cycloalkenyl, alkaryl, and aralkyl groups. Cycloaliphatic groups may contain both cyclic portions and noncyclic portions. Hydrocarbylene means a hydrocarbyl group or any of the described subsets having more than one valence, such as alkylene, alkenylene, alkynylene, arylene, cycloalkylene, cycloalkenylene, alkarylene and aralkylene. As used herein, percent by weight or parts by weight refer to, or are based on, the weight or the compositions unless otherwise specified.

The term “monofunctional” refers to 1,1-disubstituted alkene compounds having only one core unit. The core unit is represented by the combination of the carbonyl groups and the alkylene groups bonded to the 1 carbon atom.

The term “difunctional” refers to 1,1-disubstituted alkenes compounds having two core formulas (containing a reactive alkene functionality) bound through a hydrocarbylene linkage between one oxygen atom on each of two core formulas.

The term “multifunctional” refers to 1,1-disubstituted alkene compounds having more than one core unit (such as reactive alkene functionality) which may form a chain through a hydrocarbylene linkage between one heteroatom (oxygen atom) or direct bond on each of two adjacent core formulas.

The term “ketal” refers to a molecule having a ketal functionality; i.e., a molecule containing a carbon bonded to two —OR groups, where O is oxygen and R represents any alkyl group or hydrogen.

The term “volatile” refers to a compound which is capable of evaporating readily at normal temperatures and pressures.

The term “non-volatile” refers to a compound which is not capable of evaporating readily at normal temperatures and pressures.

The term “stabilized” (e.g., in the context of “stabilized” 1,1-disubstituted alkene compounds or compositions comprising the same) refers to the tendency of the compounds (or their compositions) to substantially not polymerize with time, to substantially not harden, form a gel, thicken, or otherwise increase in viscosity with time, and/or to substantially show minimal loss in cure speed (i.e., cure speed is maintained) with time.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

The present teachings relate to a general synthetic route for producing carbonyl-substituted alkenes. The process may relate to the methylenation of starting carbonyl compounds. The process may be a direct route to 1,1-disubstited alkenes, thereby eliminating an intermediate diol and/or a diol cracking step. The process may include contacting a catalyst with a starting carbonyl compound adding formaldehyde and refluxing the mixture to form a carbonyl-substituted alkene. The process may be a Mannich-type reaction. The carbonyl-substituted alkenes resulting from the process may include methylene malonates, methylene dimalonamides, methylene keto malonamides, methylene diketones, methylene keto esters, and the like.

The starting carbonyl compound for the process may be a dicarbonyl compound containing one or more ester groups, one or more keto groups, one or more amide groups, or a combination thereof. The starting carbonyl compound may be a liquid. The starting carbonyl compound may be a solid capable of dissolving or being suspended within a solvent. The starting carbonyl compound may have a melting point at or below the temperature at which the reaction will be subjected. For example, the melting point of the starting carbonyl compound may be about 80° C. or less. The starting carbonyl compound may be a diester, diketone, diamide, ketoester, ketoamide, or ester amide, for example. The starting carbonyl compound may be a hydrocarbyl malonate. The hydrocarbyl group may be an alkyl, cycloalkyl, or polyether group. The dicarbonyl compound may be a diketone with one or more aryl-substituted alkyl groups. The starting carbonyl compound may correspond to the formula:

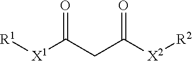

wherein X¹ and X², separately in each occurrence, are an oxygen atom or a direct bond; and wherein R¹ and R², separately in each occurrence, are hydrocarbyl groups that are the same or different. The starting carbonyl compound may include ester groups corresponding to the formula:

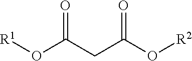

wherein R¹ and R², separately in each occurrence, are hydrocarbyl groups that are the same or different. The starting carbonyl compound may include keto groups corresponding to the formula:

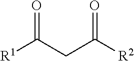

wherein R¹ and R², separately in each occurrence, are hydrocarbyl groups that are the same or different. The starting carbonyl compound may include one or more ester groups and one or more keto groups corresponding to the formula:

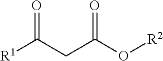

wherein R¹ and R², separately in each occurrence, are hydrocarbyl groups that are the same or different. The starting carbonyl compound may include one or more amide groups corresponding to the formula:

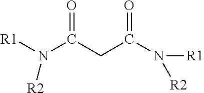

wherein R1 and R2, separately in each occurrence, is a hydrogen or a hydrocarbyl group optionally substituted with one or more heteroatoms. Other combinations of ester groups, keto groups, and amide groups are also contemplated. The starting carbonyl compound may be selected from 1-phenylbutane-1,3-dione; 1,3-diphenylpropane-1,3-dione; diethyl malonate; dicyclohexyl malonate; ethyl cyclohexyl malonate; or any other malonate.

The process includes contacting the starting carbonyl compound with a catalyst. The catalyst may be a heterogeneous catalyst. The catalyst may be selected based on its ability to be easily separated out by processes such as crystallization at low temperatures, separation via extraction with water (e.g., liquid-liquid extraction), and the like. For example, the catalyst may be selected based on its functionality, which may make the catalyst insoluble in the reaction mixture. The catalyst may be prepared by reacting an acid with a base. The catalyst may be an amine salt catalyst. The amine catalyst may be a sterically hindered ammonium cation coupled with an acid. Therefore, the base may be a sterically hindered ammonium cation. The base may be a secondary ammonium cation. The acid may be an intermediate Bronsted acid. The acid may have a Bronsted acidity of about 2 or less, about 1 or less, or about 0 or less. The acid may have a Bronsted acidity of about −6 or greater, about −4 or greater, or about −2 or greater. The acid may have a pKa of about 2 or less, about 1 or less, or about 0 or less. The acid may have a pKa of about −6 or greater, about −4 or greater, or about −2 or greater. For example, the acid may be selected from trifluoroacetic acid, sulfuric acid, methanesulfonic acid, or acetic acid. The amine salt catalyst may be selected from diisopropylammonium trifluoroacetate, diisopropylammonium acetate, or diisopropylammonium methanesulfonate, for example.

The catalyst may be supplied as a pre-isolated salt. The salt may be prepared by reacting an acid with a base to obtain a solid, which is purified by crystallization in methanol. For example, a Mannich salt may be prepared by reacting trifluoroacteic acid with diisopropylamine to obtain a white-yellow solid, which is then purified by crystallization in methanol to obtain white crystals. The pre-isolated salt may be added to the starting carbonyl compound, followed by formaldehyde, and heated for the reaction to occur. The catalyst may be supplied in a catalytic amount. The catalyst may be supplied in an amount of about 25 mol % or less, about 20 mol % or less, about 10 mol % or less, about 8 mol % or less, or about 7 mol % or less based on the starting dicarbonyl compound.

The catalyst may be made in-situ. The starting carbonyl compound may be dissolved or suspended in a solvent. The base of the salt may be added to the solvent and starting carbonyl compound. The acid of the salt may be added to the solvent and starting carbonyl compound. Adding the base and the acid may result in forming the salt in-situ. An exotherm may be observed with the addition of the components of the catalyst and/or the in-situ formation of the catalyst. The solvent into which the starting carbonyl compound is suspended or dissolved may be a polar aprotic solvent. The solvent may have a boiling point of about 80° C. or greater, about 90° C. or greater, or about 95° C. or greater. The solvent may have a boiling point of about 120° C. or less, about 110° C. or less, or about 105° C. or less. The solvent may be selected from tetrahydrofuran (THF), methyl tetrahydrofuran, dimethoxyethane, or diethoxyethane. The catalyst may be supplied in a stoichiometric amount. The acid may be supplied in slight excess.

The process further includes adding formaldehyde or a source thereof to the catalyst and starting carbonyl compound to form a mixture. Exemplary sources of formaldehyde include formaldehyde, trioxane, formalin, or paraformaldehyde. The source of formaldehyde may be substantially free of methanol, water, or both. The formaldehyde may be added in an amount of about 2:1 or greater, about 2.15:1 or greater, or about 2.25:1 or greater moles of formaldehyde to moles of the starting dicarbonyl compound. The formaldehyde may be added in an amount of about 3:1 or less, about 2.85:1 or less, or about 2.75:1 or less moles of formaldehyde to moles of the starting dicarbonyl compound. The formaldehyde may be added in an amount of about 0.75 equivalents or greater, about 1 equivalent or greater, or about 2.0 equivalents or greater. The formaldehyde may be added in an amount of about 4 equivalents or less, about 3 equivalents or less, or about 2.1 equivalents or less.

The process may be accomplished in a continuous process. The reaction may be accomplished without the addition of a solvent. The reaction step may be accomplished at atmospheric pressure. The mixture of starting dicarbonyl compound, catalyst, and source of formaldehyde may then be heated. The reaction may be heated to about 65° C. or greater, about 70° C. or greater, or about 80° C. or greater. The reaction may be heated to about 120° C. or less, about 110° C. or less, or about 100° C. or less. The heat applied to the reaction may depend up on how the catalyst is supplied. For example, if the catalyst is supplied as a pre-isolated salt, higher temperatures may be employed. Such temperatures may be about 80° C. or greater, or about 100° C. or less. If the catalyst is supplied as an in-situ salt in the presence of a solvent, lower temperatures may be employed. For example, the reaction may occur at about 68° C. The reaction may be refluxed at this heating step for about 8 hours or less, about 6 hours or less, or about 5 hours or less. The reaction may be refluxed at this heating step for about 0.5 hours or more, about 1 hour or more, or about 2 hours or more. The reaction may be monitored by 1H NMR and/or GC-MS to check for conversion to the desired product. The process may achieve a molar yield of about 40% or greater, about 45% or greater, or about 50% or greater. The process may achieve a molar yield of about 80% or less, about 75% or less, or about 70% or less. The conversion of the limiting reagent (e.g., the malonate) may be about 85% or greater, about 90% or greater, about 95% or greater, about 97% or greater, or about 99% or greater.

At the end of the reaction, any solvent may be removed. The removal of solvent may be performed under reduced pressure, such as by a rotary evaporator. Distillation may be performed to separate the desired product from the mixture. The isolated product or the crude mixture may be taken up in ether or ethyl acetate and extracted with water, brine, or both. The organic solution obtained may be dried, such as over sodium sulfate.

The process may be exemplified by the following reaction cycle:

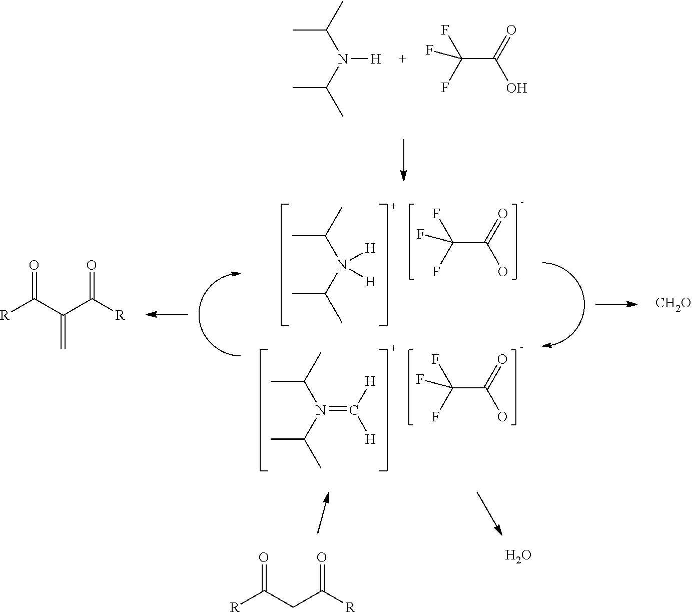

where R, separately in each occurrence, may be an alkyl group, an alkoxy group, an amine group, or a combination thereof.

The composition resulting from the processes disclosed may contain any carbonyl-substituted alkene. The process may result in monofunctional and/or polyfunctional monomers. These monomers may be produced without Michael addition. For example, the composition resulting from the process may contain 1,1-dicarbonyl substituted ethylene compounds. 1,1-dicarbonyl substituted ethylene compounds refer to compounds having a carbon with a double bond attached thereto and which is further bonded to two carbonyl carbon atoms. Exemplary compounds are shown in Formula 1:

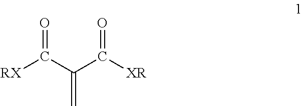

wherein R is a hydrocarbyl group which may contain one or more heteroatoms and X is oxygen or a direct bond (such as a methylene β-ketoester). Exemplary classes of 1,1-dicarbonyl substituted ethylenes are the methylene malonates, methylene beta-keto ester or diketones. Methylene malonates are exemplified by Formula 2:

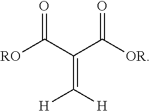

R may be independently alkyl, alkenyl, C₃-C₉ cycloalkyl, heterocyclyl, alkyl heterocyclyl, aryl, aralkyl, alkaryl, heteroaryl, or alkheteroaryl, or polyoxyakylene, or a 5-7 membered cyclic or heterocyclic ring. R may be independently C₁-C₁₅ alkyl, C₂-C₁₅ alkenyl, C₃-C₉ cycloalkyl, C₂₋₂₀ heterocyclyl, C₃₋₂₀ alkheterocyclyl, C₆₋₁₈ aryl, C₇₋₂₅ alkaryl, C₇₋₂₅ aralkyl, C₅₋₁₈ heteroaryl or C₆₋₂₅ alkyl heteroaryl, or polyoxyalkylene, or a 5-7 membered cyclic or heterocyclic ring. The recited groups may be substituted with one or more substituents, which do not interfere with the reactions disclosed herein. Exemplary substituents include halo alkylthio, alkoxy, hydroxyl, nitro, azido, cyano, acyloxy, carboxy, or ester. R may be independently C₁-C₁₅ alkyl, C₃-C₆ cycloalkyl, C₄₋₁₈ heterocyclyl, C₄₋₁₈ alkheterocyclyl, C₆₋₁₈ aryl, C₇₋₂₅ alkaryl, C₇₋₂₅ aralkyl, C₅₋₁₈ heteroaryl or C₆₋₂₅ alkyl heteroaryl, or polyoxyalkylene. R may be independently a C₁₋₄ alkyl. R may be independently methyl or ethyl. R may be the same for each ester group on the 1,1-dicarbonyl substituted ethylenes. Exemplary compounds are dimethyl, diethyl, ethylmethyl, dipropyl, dibutyl, diphenyl, and ethyl-ethylgluconate malonates; or dimethyl and diethyl methylene malonate (R is either methyl or ethyl).

Another exemplary class of carbonyl-substituted alkenes may include one or more amine groups. One or both of the —XR groups of Formula 1 above may instead be:

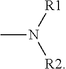

The carbonyl-substituted alkenes may then form methylene dimalonamides, methylene ketomalonamides, and the like. For example, a carbonyl-substituted alkene having two or more amine groups may correspond to Formula 3:

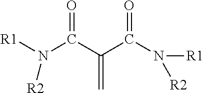

where R1 and R2, separately in each occurrence may be a hydrogen or a hydrocarbyl group with one or more heteroatoms. A carbonyl-substituted alkene, such as a mixed functionality monomalonamide, having one or more amine groups may correspond to Formula 4:

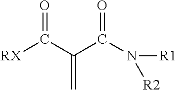

where R is a hydrocarbyl group which may contain one or more heteroatoms; X is oxygen or a direct bond; and R1 and R2, may be independently a hydrogen or a hydrocarbyl group with one or more heteroatoms. The resulting carbonyl-substituted alkene may, for example, be selected from:

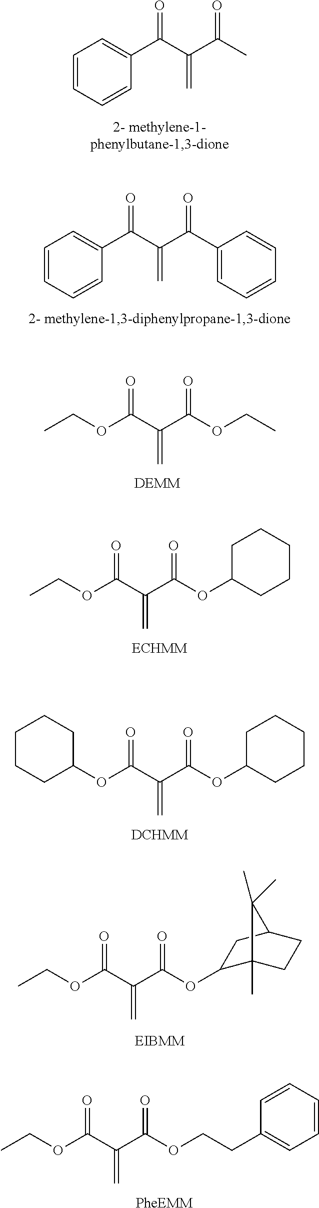

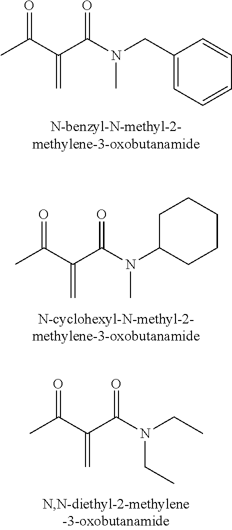 [SMILES: C=C(C(C)=O)C(=O)N(C)Cc1ccccc1 ; C=C(C(C)=O)C(=O)N(C)C1CCCCC1 ; C=C(C(C)=O)C(=O)N(CC)CC]

The carbonyl-substituted alkene compounds disclosed herein may exhibit a sufficiently high purity so that they can be polymerized. The purity of the carbonyl-substituted alkenes may be sufficiently high so that about 70 mole percent or more, about 80 mole percent or more, about 90 mole percent or more, about 95 mole percent or more, or about 99 mole percent or more of the carbonyl-substituted alkene is converted to polymer during a polymerization process. The purity of the carbonyl-substituted alkenes is about 96 mole percent or greater, about 97 mole percent or greater, about 98 mole percent or greater, about 99 mole percent or greater, or about 99.5 mole percent or greater, based on the total weight of the carbonyl-substituted alkenes. The concentration of any impurities containing a dioxane group may be about 2 mole percent or less, about 1 mole percent or less, about 0.2 mole percent or less, or about 0.05 mole percent or less, based on the total weight of the carbonyl-substituted alkenes. The total concentration of any impurity having the alkene group replaced by an analogous hydroxyalkyl group (e.g., by a Michael addition of the alkene with water) may be about 3 mole percent or less, about 1 mole percent or less, about 0.1 mole percent or less, or about 0.01 mole percent or less, based on the total moles in the carbonyl-substituted alkenes.

The one or more 1,1-dicarbonyl substituted-1-ethylenes prepared may be isolated using any known processes for recovering such products, see for example Malofsky et al., U.S. Pat. No. 8,609,8985; Mardirossian, U.S. Pat. No. 8,884,051 and Malofsky et al., U.S. Pat. No. 9,108,914. The desired products are separated from a variety of by-products, side reaction products and impurities. A variety of separation processes or operations may be utilized. Exemplary separation processes include a series of condensation and distillation steps. Exemplary separation units include, hot condensers, condensers, vacuum distillation apparatuses, simple distillation apparatuses and/or fractional distillation apparatuses. Liquid-liquid extraction processes may be employed. Such process may be helpful for removal of salt impurities, for example. In some exemplary embodiments, the separation techniques may be employed at atmospheric pressure, under vacuum, or under elevated pressure, in accordance with sound engineering principles.

The compositions disclosed may be utilized to prepare homo and copolymers. The higher purity of the compositions provides greater control of the homo and copolymers so that desired molecular weights and polydispersities can be prepared. High amounts of 1,1-disubstituted alkenes in the polymers function to plasticize the polymers, which may be undesirable for many applications. Thus, lower amounts of 1,1-disubstituted alkenes may result in better control polymer properties. The polymers contain about 3 weight percent or less of 1,1-disubstituted alkenes, about 2 weight percent or less of 1,1-disubstituted alkenes, or about 1 weight percent or less of 1,1-disubstituted alkenes. The polymers prepared may have molecular weights of about 5,000 Daltons or greater, molecular weights of about 10,000 Daltons or greater or molecular weights of about 500,000 Daltons or greater. The polymers may have molecular weights of about 1,000,000 Daltons or less or about 100,000 or less. The polymers prepared may have polydispersities of about 1 or greater. The polymers may have polydispersities of about 3 or less, about 2 or less or about 1.1 or less.

The polymerizable compositions disclosed herein can be polymerized by exposing the composition to free radical polymerization conditions or to anionic polymerization conditions. Free radical polymerization conditions are well known to those skilled in the art such as disclosed in Sutoris et al., U.S. Pat. No. 6,458,956. In certain embodiments, the polymerizable compositions are exposed to anionic polymerization conditions. The polymerizable compositions are contacted with any anionic polymerization initiator or with any nucleophilic material. As 1,1-disubstituted alkenes, which may be highly electrophilic, contacts any nucleophilic material, this can initiate anionic polymerization. Anionic polymerization is commonly referred to as living polymerization because the terminal portion of the polymeric chains are nucleophilic and will react with any unreacted 1,1-disubstituted alkenes. Thus, the polymerizable composition will continue until all available unreacted 1,1-disubstituted alkenes polymerize or the polymerizing mixture is subjected to a quenching step. In a quenching step the mixture is contacted with an acid which terminates the polymeric chain ends and stops further polymerization. The polymerization can proceed at any reasonable temperature including at ambient temperatures, from about 20° C. to about 35° C., depending on ambient conditions. The polymerization can be performed in bulk, without a solvent or dispersant, or in a solvent or dispersant.

According to certain embodiments, a suitable polymerization initiator can generally be selected from any agent that can initiate polymerization substantially upon contact with a selected polymerizable composition. In certain embodiments, it can be advantageous to select polymerization initiators that can induce polymerization under ambient conditions and without requiring external energy from heat or radiation. In embodiments where the polymerizable composition comprises one or more 1,1-disubstituted alkene compounds, a wide variety of polymerization initiators can be utilized including most nucleophilic initiators capable of initiating anionic polymerization. Exemplary initiators include metal carboxylate salts, alkaline earth carboxylate salts, amines, halides (halogen containing salts), metal oxides, and mixtures containing such salts or oxides. Exemplary anions for such salts include anions based on halogens, acetates, benzoates, sulfur, carbonates, silicates and the like. The mixtures containing such compounds can be naturally occurring or synthetic. Specific examples of exemplary polymerization initiators for 1,1-disubstituted alkene compounds can include glass beads (being an amalgam of various oxides including silicon dioxide, sodium oxide, and calcium oxide), ceramic beads (comprised of various metals, nonmetals and metalloid materials), clay minerals (including hectorite clay and bentonite clay), and ionic compounds such as sodium silicate, sodium benzoate, and calcium carbonate. Other polymerization initiators can also be suitable including certain plastics (e.g., ABS, acrylic, and polycarbonate plastics) and glass-fiber impregnated plastics. Additional suitable polymerization initiators for such polymerizable compositions are also disclosed in Malofsky et al., U.S. Patent App. Publication No. 2015/0073110. In some embodiments, the polymerization initiator may be encapsulated using any encapsulation method compatible with the polymerization of the 1,1-disubstituted alkenes. In some embodiments, the encapsulated initiator (activation agent) may be as disclosed in Stevenson et al., U.S. Pat. No. 9,334,430.

Polymerization can be terminated by contacting the polymeric mixture with an anionic polymerization terminator. In some embodiments the anionic polymerization terminator is an acid. In some embodiments it is desirable to utilize a sufficient amount of the acid to render the polymerization mixture slightly acidic, having a pH of about 7 or less or about 6 or less. Exemplary anionic polymerization terminators include, for example, mineral acids such as methane sulfonic acid, sulfuric acid, and phosphoric acid and carboxylic acids such as acetic acid and trifluoroacetic acid.

The polymerizable compositions may be polymerized in bulk, which is in the absence of a solvent or dispersant, in a solution or in an emulsion. Polymerization in bulk can be performed by contacting the polymerizable composition which may include any of the other ingredients disclosed herein with a suitable substrate and an activator and allowing the composition to polymerize.

The polymerizable compositions may be prepared by emulsion polymerization. For example, the polymerizable compositions may be prepared by the process disclosed in Stevenson et al., U.S. Pat. No. 9,249,265. Disclosed in Stevenson et al., U.S. Pat. No. 9,249,265 is a process comprising the steps of: agitating a mixture including: about 25 weight percent or more of a carrier liquid, a surfactant (e.g., an emulsifier) and one or more monomers to form micelles of the one or more monomers in the carrier liquid, wherein the one or more monomers includes one or more 1,1-disubstituted alkenes; reacting an activator with at least one of the monomers in the micelle for initiating the anionic polymerization of the one or more monomers; and anionically polymerizing the one or more monomers. The polymerization process includes one or more surfactants for forming an emulsion having micelles or a discrete phase including a monomer (e.g., a 1,1-disubstituted alkene compound) distributed throughout a continuous phase (e.g., a continuous phase including a carrier liquid). The surfactant may be an emulsifier, a defoamer, or a wetting agent. The surfactant in some embodiments is present in a sufficient quantity so that a stable emulsion is formed by mixing or otherwise agitating a system including the monomer and carrier liquid. The surfactants according to the teachings herein include one or more surfactants for improving the stability of emulsion (i.e., for improving the stability of the dispersed phase in the carrier liquid phase). The surfactant and/or the amount of surfactant is selected so that all of the monomer micelles are covered by a layer of the surfactant. The surfactant may include an amphoteric surfactant, a nonionic surfactant, or any combination thereof. The surfactant is free of anionic surfactants during the polymerization process. One example of a surfactant (e.g., an emulsifier) is an ethoxylate, such as an ethoxylated diol. For example, the surfactant may include 2,4,7,9-tetramethyl-5-decyne-4,7-diol ethoxylate. The surfactant may include a poly(alkene glycol). Another example of a surfactant is a poly(ethylene glycol)-block-poly(propylene glycol)-block-poly(ethylene glycol) copolymer. Another example of a surfactant is a surfactant including an alcohol, an ethoxylated alcohol, or both. For example, the surfactant may include CARBOWET® 138 nonionic surfactant (including alkyl alcohol, polyethylene glycol, ethoxylated C₉-C₁₁ alcohols). Another example of a surfactant is a surfactant including a sorbitan, a sorbitol, or a polyoxyalkene. For example, the surfactant may include sorbitan monopalmitate (nonionic surfactant). Other examples of surfactants include branched polyoxyethylene (12) nonylphynyl ether (IGEPAL® CO-720) and poly(ethylene glycol) sorbitol hexaoleate (PEGSH). The amount of the surfactant (e.g., the amount of the emulsifier) in some embodiments is sufficient to form a layer that substantially encapsulates the monomer and subsequent polymer particles. The amount of surfactant is sufficient so that the discrete phase has a diameter of about 10 mm or less, about 1 mm or less, about 300 μm or less, or about 100 μm or less. The amount of the surfactant is sufficient so that the discrete phase has a diameter of about 0.01 μm or more, about 0.1 μm or more, about 1 μm or more, about 10 μm or more, or about 50 μm or more. The concentration of the surfactant may be about 0.001 weight percent or more, about 0.01 weight percent or more, about 0.1 weight percent or more, or about 0.5 weight percent or more, based on the total weight of the emulsion. The concentration of the surfactant may be about 15 weight percent or less, about 10 weight percent or less, and about 6 weight percent or less, or about 3 weight percent or less, based on the total weight of the emulsion. The weight ratio of the surfactant to the total weight of the monomer and polymer in the emulsion (e.g., at the end of the polymerization process) is about 0.0001 or more, about 0.002 or more, about 0.005 or more, or about 0.01 or more. The weight ratio of the surfactant to the total weight of the monomer and polymer in the emulsion (e.g., at the end of the polymerization process) is about 5 or less (i.e., about 5:1 or less), about 1 or less, about 0.5 or less, or about 0.1 or less. The carrier liquid is in some embodiments is water. The polymerization process may include a step of applying shear forces or sonication to a mixture including at least the surfactant and the carrier fluid for forming an emulsion. For example, the process may include stirring or otherwise agitating the mixture for creating the emulsion.

The polymerizable compositions disclosed herein may be polymerized in solution via anionic polymerization processes. In some embodiments, the polymerizable compositions may be polymerized utilizing the method disclosed in Palsule et al., U.S. Pat. No. 9,279,022. According to the process disclosed in Palsule et al., U.S. Pat. No. 9,279,022 the process comprises the steps of mixing one or more 1,1-disubstituted alkenes and a solvent; adding an activator; reacting the activator with the one or more 1,1-disubstituted alkenes to initiate the anionic polymerization of the one or more 1,1-disubstituted alkenes; and anionically polymerizing the one or more 1,1-disubstituted alkenes to form a polymer. The concentration of the monomer in the solution polymerization process may be sufficiently low so that after polymerization, the solution can flow. If the concentration of the monomer is too high, the solution becomes too viscous at the end of the polymerization process and the solution may be difficult to handle. The concentration of the monomer in the solution polymerization process may be sufficiently high so that the polymerization process is economical. The one or more monomers is present at a concentration of about 0.5 weight percent or more, about 2 weight percent or more, about 5 weight percent or more, or about 8 weight percent or more, based on the total weight of the solvent and monomer. The one or more monomers may be present at a concentration of about 90 weight percent or less, about 75 weight percent or less, about 50 weight percent or less, about 30 weight percent or less, or about 20 weight percent or less. If the monomer is added at multiple times (such as continuous and/or sequential monomer addition), it will be appreciated that the amount of the one or more monomers refers to the total amount of monomer and polymer and by-products of the monomer that are present when the addition of monomer has been completed. The polymerization process includes one or more solvents selected so that the monomer and solvent form a single phase. The solvent typically does not chemically react with the other components of the solution polymerization system during the polymerization process. For example, the solvent does not react with the monomer. As another example, the solvent does not react with the activator. Exemplary solvents are organic solvents, or mixtures of organic solvents. Such solvents, or solvent mixtures typically are in a liquid state at the reaction temperature(s) (e.g., during activation and/or during polymerization. The pressure of the solvent (e.g., organic solvent) and of the monomer at the polymerization temperature should be sufficiently low so that the risk of the reactor failing from over-pressure is reduced or eliminated. For example, the partial pressure of the solvent, of the monomer, or both, at the polymerization temperature may be about 500 Torr or less, about 200 Torr or less, about 50 Torr or less, or about 5 Torr or less. It may be desirable for the solvent to be substantially or entirely free of any solvent that may react with the monomer via Michael addition. However, by selecting reaction conditions so that the polymerization reaction is sufficiently fast, it may be possible to employ such monomers in the solvent polymerization process. For example, by selecting parameters such as monomer feed rates, reaction temperature, monomer type, and pH, it may be possible to employ a solvent including or consisting of a protic solvent, such as an alcohol. The solution polymerization may be initiated using an activator capable of initiating anionic polymerization of the 1,1-disubstituted alkene containing compound. The solvent and/or one or more of the monomers (e.g., the 1,1-disubstituted alkene compounds) may further contain other components to stabilize the monomer prior to exposure to polymerization conditions or to adjust the properties of the final polymer for the desired use. Prior to the polymerization reaction, one or more inhibitors may be added to reduce or prevent reaction of the monomer. Such inhibitors may be effective in preventing anionic polymerization of the monomer, free radical polymerization of the monomer, reaction between the monomer and other molecules (such as water), or any combination thereof.

The polymerization processes disclosed may include a step of applying shear forces to a mixture including at least the monomer and the solvent or carrier. For example, the process may include stirring or otherwise agitating the mixture for creating the solution or emulsion, for dispersing or removing a precipitated polymer, for controlling thermal gradients, or any combination thereof. The polymerization processes may include a reaction temperature at which the partial pressure of the solvent is generally low. For example, the partial pressure of the solvent and/or the monomer may be about 400 Torr or less, about 200 Torr or less, about 100 Torr or less, about 55 Torr or less, or about 10 Torr or less. The reaction temperature is about 80° C. or less, about 70° C. or less, about 60° C. or less, about 55° C. or less, about 45° C. or less, about 40° C. or less, or about 30° C. or less. The reaction temperature typically is sufficiently high that the solvent or carrier liquid and the monomer are in a liquid state. For example, the reaction temperature may be about −100° C. or more, about −80° C. or more, about −30° C. or more, or about 10° C. or more. When polymerizing a 1,1-disubstituted alkene compound, it may be desirable to add one or more acid compounds to the solution, to the monomer, or both, so that the initial pH of the solution is about 7 or less, about 6.8 or less, about 6.6 or less, or about 6.4 or less. The polymerization process may be stopped prior to the completion of the polymerization reaction or may be continued until the completion of the polymerization reaction. In some embodiments, the reaction rate is sufficiently high, and/or the reaction time is sufficiently long so that the polymerization reaction is substantially complete.

The conversion of the monomer to polymer may be about 30 weight percent or more, about 60 weight percent or more, about 90 weight percent or more, about 95 weight percent or more, or about 99 weight percent or more. The conversion of monomer to polymer may be about 100 weight percent or less.

The polymerizable compositions may further contain other components to stabilize the compositions prior to exposure to polymerization conditions or to adjust the properties of the final polymer for the desired use. For example, in certain embodiments, a suitable plasticizer can be included with a reactive composition. Exemplary plasticizers are those used to modify the rheological properties of adhesive systems including, for example, straight and branched chain alkyl-phthalates such as diisononyl phthalate, dioctyl phthalate, and dibutyl phthalate, trioctyl phosphate, epoxy plasticizers, toluene-sulfamide, chloroparaffins, adipic acid esters, sebacates such as dimethyl sebacate, castor oil, xylene, 1-methyl-2-pyrrolidone and toluene. Commercial plasticizers such as HB-40 partially hydrogenated terpene manufactured by Solutia Inc. (St. Louis, Mo.) can also be suitable.

One or more dyes, pigments, toughening agents, impact modifiers, rheology modifiers, natural or synthetic rubbers, filler agents, reinforcing agents, thickening agents, opacifiers, inhibitors, fluorescence markers, thermal degradation reducers, thermal resistance conferring agents, surfactants, wetting agents, or stabilizers can be included in a polymerizable system. For example, thickening agents and plasticizers such as vinyl chloride terpolymer (comprising vinyl chloride, vinyl acetate, and dicarboxylic acid at various weight percentages) and dimethyl sebacate respectively, can be used to modify the viscosity, elasticity, and robustness of a system. In certain embodiments, such thickening agents and other compounds can be used to increase the viscosity of a polymerizable system from about 1 to 3 cPs to about 30,000 cPs, or more.

According to certain embodiments, stabilizers can be included in the polymerizable compositions to increase and improve the shelf life and to prevent spontaneous polymerization. Generally, one or more anionic polymerization stabilizers and or free-radical stabilizers may be added to the compositions. Anionic polymerization stabilizers are generally electrophilic compounds that scavenge bases and nucleophiles from the composition or growing polymer chain. The use of anionic polymerization stabilizers can terminate additional polymer chain propagation. Exemplary anionic polymerization stabilizers are acids, exemplary acids are carboxylic acids, sulfonic acids, phosphoric acids and the like. Exemplary stabilizers include liquid phase stabilizers (e.g., methanesulfonic acid (“MSA”)), and vapor phase stabilizers (e.g., trifluoroacetic acid (“TFA”)). Free-radical stabilizers include, for example, phenolic compounds (e.g., 4-methoxyphenol or mono methyl ether of hydroquinone (“MeHQ”) and butylated hydroxy toluene (BHT)). Stabilizer packages for 1,1-disubstituted alkenes are disclosed in Malofsky et al., U.S. Pat. No. 8,609,885 and Malofsky, U.S. Pat. No. 8,884,051. Additional free radical polymerization inhibitors are disclosed in Sutoris et al., U.S. Pat. No. 6,458,956. Generally, only minimal quantities of a stabilizer are needed and, in certain embodiments only about 150 parts-per-million or less can be included. In certain embodiments, a blend of multiple stabilizers can be included such as, for example a blend of anionic stabilizers (MSA) and free radical stabilizers (MeHQ). The one or more anionic polymerization stabilizers are present in sufficient amount to prevent premature polymerization. The anionic polymerization stabilizers are present in an amount of about 0.1 part per million or greater based on the weight of the composition, about 1 part per million by weight or greater or about 5 parts per million by weight or greater. The anionic polymerization stabilizers are present in an amount of about 1000 parts per million by weight or less based on the weight of the composition, about 500 parts per million by weight or less or about 100 parts per million by weight or less. The one or more free radical stabilizers are present in sufficient amount to prevent premature polymerization. The free radical polymerization stabilizers are present in an amount of about 1 parts per million or greater based on the weight of the composition, about 5 parts per million by weight or greater or about 10 parts per million by weight or greater. The free radical polymerization stabilizers are present in an amount of about 5000 parts per million by weight or less based on the weight of the composition, about 1000 parts per million by weight or less or about 500 parts per million by weight or less.

The polymerizable compositions and polymers disclosed herein may be utilized and a number of applications. Exemplary applications include adhesives, sealants, coatings, components for optical fibers, potting and encapsulating materials for electronics, resins and pre-polymers as raw materials in other systems, and the like.

The polymerizable compositions exhibit a number of advantageous properties including rapid reactivity, room or low temperature reactivity, tailorable rheological characteristics, and the like. Polymers prepared from the polymerizable compositions exhibit a number of advantageous properties including for example, high glass transition temperature, high degradation temperature, high heat resistance, high stiffness and modulus, good rigidity and the like.

Other components commonly used in curable compositions may be used in the compositions of this invention. Such materials are well known to those skilled in the art and may include, for example, ultraviolet stabilizers and antioxidants and the like. The compositions described herein may also contain durability stabilizers known in the art. In some embodiments, durability stabilizers are alkyl substituted phenols, phosphites, sebacates and cinnamates.

The process disclosed allows the preparation of 1,1-disubstituted alkenes at higher yields than previously possible. The product yield may be about 90 percent or greater, about 93 percent or greater, or about 95 percent of greater.

Molecular weights as described herein are number average molecular weights which may be determined by Gel Permeation Chromatography (also referred to as GPC) using a polymethylmethacrylate standard.

The processes disclosed may further comprise any one or more of the features described in this specification in any combination, including the embodiments and examples provided in the specification, and includes the following features: the process may be a Mannich-type reaction; the formaldehyde, paraformaldehyde, or formalin may be added in an amount of about 2.0 to about 2.1 equivalents; the dicarbonyl compound may be a diester, diketone, diamide, ketoester, ketoamide, or ester amide; the dicarbonyl compound may be a hydrocarbyl malonate, where the hydrocarbyl group is an alkyl, cycloalkyl, polyether group; or a diketone with one or more aryl-substituted alkyl groups; the dicarbonyl compound may be selected from 1-phenylbutane-1,3-dione; 1,3-diphenylpropane-1,3-dione; diethyl malonate; dicyclohexyl malonate; or ethyl cyclohexyl malonate; or any other malonate; the amine salt catalyst may be prepared by reacting an acid with a base; the acid may have a Bronsted acidity of about 2 to about −6; the base may be a sterically hindered ammonium cation; the base may be a secondary ammonium cation; the acid may have a pKa of about 2 to about −6; the acid may be selected from trifluoroacetic acid, sulfuric acid, methanesulfonic acid, or acetic acid; the amine salt catalyst may be diisopropylammonium trifluoroacetate, diisopropylammonium acetate, or diisopropylammonium methanesulfonate; the amine salt catalyst may be supplied in a catalytic amount; the amine salt catalyst may be provided in an amount of about 25 mol % or less based on the dicarbonyl compound; the amine salt catalyst may be provided in an amount of about 7 mol % or less based on the dicarbonyl compound; the process may occur in the absence of a solvent; the amine salt catalyst may be supplied in a stoichiometric amount; the process may include dissolving the dicarbonyl compound in a solvent; the solvent may be a polar aprotic solvent; the solvent may be selected from tetrahydrofuran (THF), methylTHF, dimethoxyethane or diethoxyethane; the mixture may be heated to a temperature of about 50° C. or greater or about 80° C. or greater; the mixture may be heated to a temperature of about 120° C. or less; the process may have a reaction time of about 5 hours or less; the process may have a reaction time of about 3 hours or less; the process may achieve a molar yield of about 45% or greater; the process may achieve a molar yield of about 75% or greater; the conversion of the limiting reagent (e.g., malonate) may be greater than about 85%; the carbonyl-substituted alkene may be shown in the formula:

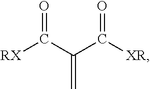

where R, separately in each occurrence, may be a hydrocarbyl group with one or more heteroatoms; and X, separately in each occurrence, may be oxygen or a direct bond; the carbonyl-substituted alkene may be shown in the formula:

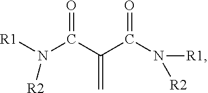

where R1 and R2, separately in each occurrence, may be a hydrogen or a hydrocarbyl group with one or more heteroatoms; the carbonyl-substituted alkene may be selected from:

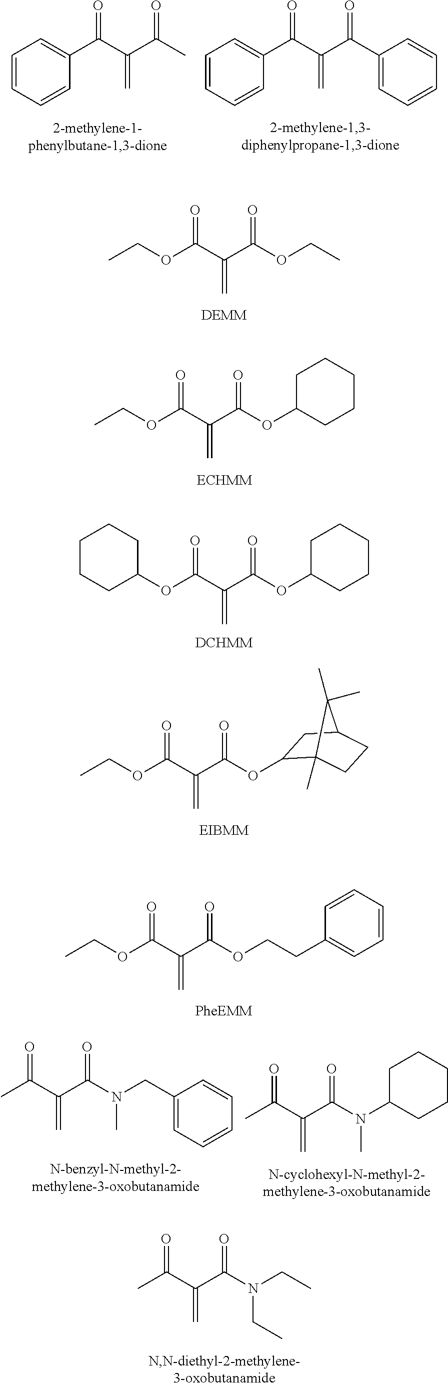

a carbonyl-substituted alkene formed by the process as described herein; or a polymer prepared from the carbonyl-substituted alkenes described herein.

All publications and patents cited herein are incorporated by reference in their entirety for all purposes.

### ILLUSTRATIVE EMBODIMENTS

The following examples are provided to illustrate the disclosed compositions but are not intended to limit the scope thereof. All parts and percentages are by weight unless otherwise stated.

### Example 1

A starting dicarbonyl compound, in an amount of 1.0 equivalent, is charged to a three-necked round bottom flask fitted with a thermocouple and a reflux condenser. 3000 ppm butylated hydroxytoluene is added. A pre-isolated Mannich salt, prepared by reacting an acid (trifluoroacetic acid) with a base (diisopropylamine) to obtain a white-yellow solid, which is purified by crystallization in methanol to obtain white crystals, is added to the solution in an amount of 7.0 mol % with respect to the starting dicarbonyl compound. 2.0-2.1 equivalents of formaldehyde, paraformaldehyde, or formalin are then added. The reaction is heated at about 85° C. to about 90° C. for about 2 to about 5 hours. The reaction is monitored by ¹H NMR and GC-MS to check for conversion to desired product. Distillation is performed at the end of the reaction to isolate the desired product from the mixture. The isolated product is then taken up in ethyl acetate and extracted with water three times and then with brine one time. The organic solution obtained in then dried over sodium sulfate. The solvent is removed under reduced pressure.

This process allows for the use of a Mannich salt in a catalytic amount. The reaction is solvent-free. The process allows for the following materials, among others, to be synthesized:

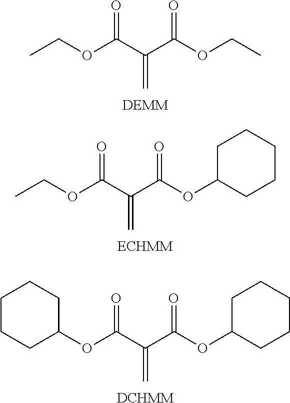

### Example 2

A starting dicarbonyl compound, in an amount of 1.0 equivalent, is charged in a three-necked round bottom flask fitted with a thermocouple and a reflux condenser. Tetrahydrofuran (THF) is added to the flask and the substrate is dissolved or suspended. To this solution, 1.0 equivalents of base is added, followed by the addition of the acid in 1.1 equivalents, to make the salt in-situ. An exotherm is observed, and the reaction is stirred until the flask returns to ambient temperature. Paraform, in an amount of 2.0-2.1 equivalents is then added to the reaction mixture. The mixture is heated to 68° C. The reaction is then refluxed at elevated temperature, about 68° C., for about 2 to about 5 hours. The reaction is monitored by ¹H NMR and GC-MS to check for conversion to desired product. At the end of the reaction, THF is removed under reduced pressure. The crude mixture is taken up in ether or ethyl acetate and extracted with water three times and then with brine one time. The organic solution obtained is then dried over sodium sulfate. The solvent is removed under reduced pressure using a rotary evaporator, and the product obtained is distilled or crystallized using ethyl acetate as the solvent.

The process allows for the use of a Mannich salt in stoichiometric amount using THF as a solvent. The process allows for the following materials, among others, to be synthesized:

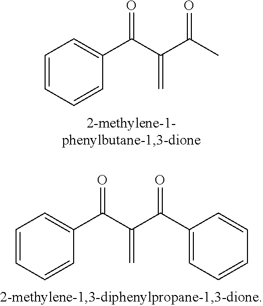 [SMILES: C=C(C(C)=O)C(=O)c1ccccc1 ; C=C(C(=O)c1ccccc1)C(=O)c1ccccc1]

Parts by weight as used herein refers to 100 parts by weight of the composition specifically referred to. Any numerical values recited in the above application include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, from 20 to 80, from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value, and the highest value enumerated are to be considered to be expressly stated in this application in a similar manner. Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of the elements, ingredients, components or steps. Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

1. A process comprising:
a) contacting an acid amine salt catalyst with a diester or ketoester compound having an alkylene group between the carbonyl groups;
b) adding formaldehyde, paraformaldehyde, formalin or trioxane in an amount of 2:1 to 3:1 moles of formaldehyde to moles of the compound to form a mixture; and
c) heating the mixture in the absence of a solvent;
wherein the process forms a diester or ketoester substituted alkene.

2. The process of claim 1, wherein the process is a Mannich-type reaction.

3. The process of claim 1, wherein the formaldehyde, paraformaldehyde, formalin or trioxane is added in an amount of 2.0 to 2.1 equivalents.

4. The process of claim 1, wherein the diester or ketoester compound is a hydrocarbyl malonate, where the hydrocarbyl group is an alkyl, cycloalkyl, polyether group.

5. The process of claim 4, wherein the diester or ketoester compound is selected from diethyl malonate; dicyclohexyl malonate; or ethyl cyclohexyl malonate.

6. The process of claim 1, wherein the amine salt catalyst is prepared by reacting an acid with a base, and wherein the acid has a Bronsted acidity of 2 to −6.

7. The process of claim 6, wherein the base is a sterically hindered ammonium cation.

8. The process of claim 6, wherein the base is a secondary ammonium cation.

9. The process of claim 6, wherein the acid has a pKa of about 2 to about 6.

10. The process of claim 6, wherein the acid is selected from trifluoroacetic acid, sulfuric acid, methanesulfonic acid, or acetic acid.

11. The process of claim 1, wherein the amine salt catalyst is diisopropylammonium trifluoroacetate, diisopropylammonium acetate, or diisopropylammonium methanesulfonate.

12. The process of claim 1, wherein the amine salt catalyst is supplied in a catalytic amount.

13. The process of claim 12, wherein the amine salt catalyst is provided in an amount of about 25 mol % or less based on the compound.

14. The process of claim 13, wherein the amine salt catalyst is provided in an amount of about 7 mol % or less based on the compound.

15. The process of claim 1, wherein the amine salt catalyst is supplied in a stoichiometric amount.

16. The process of claim 1, wherein the mixture is heated to a temperature of 80° C. or greater.

17. The process of claim 16, wherein the mixture is heated to a temperature of about 120° C. or less.

18. The process of claim 17, wherein the process has a reaction time of about 5 hours or less.

19. The process of claim 18, wherein the process has a reaction time of about 3 hours or less.

20. The process of claim 1, wherein the process achieves a conversion of the limiting reagent of about 85% or greater.

21. The process of claim 1, wherein the diester, or ketoester alkene is shown in Formula 1: 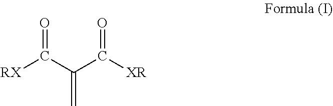
wherein each R is independently a hydrocarbyl group with one or more heteroatoms; and each X is independently oxygen, or a direct bond provided that only one X is a direct bond.

22. The process of claim 1, wherein the diester, or ketoester alkene is selected from: 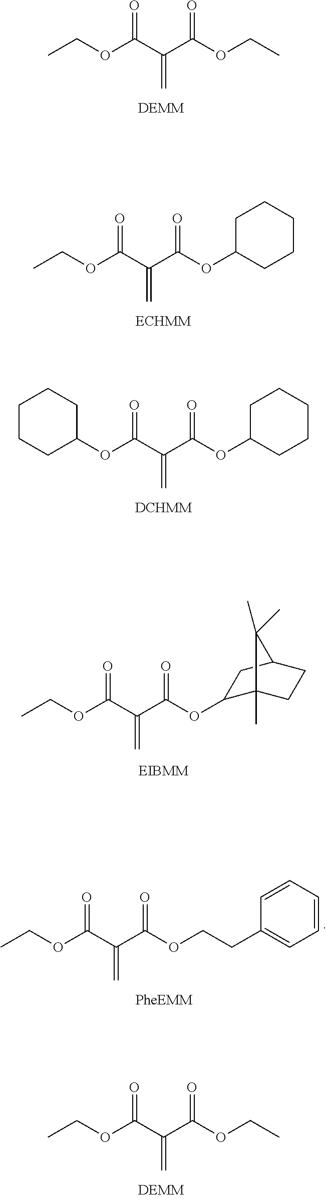 [chemical structure]
